(19) United States
(12) Patent Application Publication
(10) Pub. No.: US 2010/0120867 A1
(43) Pub. Date: May 13, 2010

(54) METHOD OF TREATMENT

(75) Inventors: Carola Friedman, New York, NY (US); Andrew Snoddy, Sussex, NJ (US)

Correspondence Address: FRANK A. SMITH;Novartis Consumer Health, Inc., 200 Kimball Drive, OTC PATENT DEPARTMENT - 5TH FLOOR, Parsippany, NJ, 07054-0622, US

(73) Assignee: Novartis AG, Basel (CH)

(21) Appl. No.: 12/692,176

(22) Filed: Jan. 22, 2010

Related U.S. Application Data
(63) Continuation of application No. 11/973,575, filed on Oct. 9, 2007, now abandoned.

(51) Int. Cl. A61K 31/4439

(52) U.S. Cl. 514/341

(57) ABSTRACT

Improvements in the treatment of patients suffering from frequent heartburn are provided by a treatment regimen comprising administration of an effective daily amount of lansoprazole or a pharmaceutically acceptable salt thereof for up to 14 days.

This is a continuation of prior application Ser. No. 11/973,575 filed Oct. 9, 2007 based on Application No. 60/850,443 filed Oct. 10, 2006.

### METHOD OF TREATMENT

### Background of the Invention

Approximately 20% of adults in the United States suffer from heartburn at least once weekly [Oliveria et al., Arch Intern Med 159(14):1592-8 (1999); Locke et al., Gastroenterol 112:1448-1456 (1997); Farup et al., Arch Intern Med 61:45-52 (2001)]. Heartburn occurs in many patients when acidic stomach contents move into the esophagus from the stomach causing a burning sensation in the chest or throat. Acid production in the stomach is stimulated when gastrin, acetylcholine, or histamine bind their respective receptors on the gastric parietal cell and induce the hydrogen/potassium ion adenosine triphosphatase proton pump (H⁺/K⁺-ATPase) to release H⁺ ions by replacing it with K⁺ [Hersey S. J., and G. Sach, Physiological Reviews 75(1):155-181 (1995); Samuelson, L. C. and K. L. Hinkle, Ann. Rev. Physio 65:383-400 (2003)].

The risk factors for frequent heartburn symptoms include obesity, spicy foods, stress, alcohol, overeating, pregnancy, family history, etc. [Oliveria, id., Bolin et al., J Gastroenterol Hepatol; 15(1):35-9 (2000)]. Treatments for heartburn include antacids, histamine-2 receptor antagonists (H₂RA), antacid/H₂RA combinations, and proton pump inhibitors (PPI). Antacids act very rapidly to neutralize acid in the esophagus for about one hour and are well suited for occasional and brief episodes of heartburn [Robinson et al. Aliment Pharmacol Ther 16:435-443 (2002), Moayyedi et al., Aliment Pharmacol Ther 17: 1215-1227 (2003)]. H₂RAs (cimetidine, ranitidine, famotidine, etc.) bind to histamine-2 receptors on gastric parietal cells and reduce acid secretion. The onset of activity is not as rapid as antacids and tolerance may develop, but the duration of action extends to about 10 hours [Moayyedi et al., id.; Gardner et al., Aliment Pharmacol Ther 16:1317-26 (2002)].

Lansoprazole, omeprazole, esomeprazole, pantoprazole and rabeprazole and their pharmaceutically acceptable salts are substituted benzimidazole derivatives which are proton pump inhibitors (hereinafter “PPI's”). PPI's covalently bind to the H⁺/K⁺-ATPase of active gastric parietal cells causing a sustained and significant inhibition of acid production lasting 24-72 hours even though they have an elimination half-life of about one hour [Hersey, id.; Stedman, C. A. M. and M. L. Barclay, Aliment Pharmacol Ther 14:963-978 (2000)]. Although PPI's have a delayed onset of activity compared to antacids and H₂RAs, comparative studies have demonstrated better symptom relief due to their efficacy in suppression of acid production [Richter et al., Arch Intern Med 160:1803-1809 (2000); Stedman, id.].

Lansoprazole, omeprazole, esomeprazole magnesium, pantoprazole and rabeprazole sodium have been approved by the Food and Drug Administration as prescription medicines for the management of duodenal ulcers, gastric ulcers, GERD, and pathologic hypersecretory conditions. Lansoprazole has additionally been approved for eradication of H. pylori in combination with clarithromycin and amoxicillin, or with amoxicillin only.

Omeprazole magnesium 20.6 mg (equivalent to 20 mg omeprazole), delayed release oral tablets, i.e. Prilosec OTC®, was approved in 2003 as an over-the counter medicine for treatment of frequent heartburn (occurring 2 or more days a week) by administration once daily for a period of 14 days. The Prilosec OTC® label states that it is not intended for immediate relief of heartburn, and that the drug may take 1 to 4 days for full effect, although some people get complete relief of symptoms within 24 hours.

### SUMMARY OF THE INVENTION

The invention comprises a method of treating or preventing frequent heartburn in a patient in need thereof which comprises administering to the patient an effective amount of lansoprazole or a pharmaceutically acceptable salt thereof per day for a period of up to 14 days.

The method of the invention has been found to provide significant reductions in the frequency and severity of daytime heartburn, and also nighttime heartburn, in patients suffering from frequent heartburn.

In particular, the invention provides a treatment regimen that achieves full effect early in the treatment regimen, i.e. within Days 1-4 and even on the second day (“Day 2”) or the first day (“Day 1”) of treating. By “full effect” is meant that the patient is rendered substantially free of heartburn during the full 24-hour day (i.e. both during daytime and at night).

The method of the invention is also found to provide a statistically significant increase in the proportion of 24-hour days during a 14-day course of therapy that a randomized treatment group administered lansoprazole or its pharmaceutically acceptable salt is substantially free of heartburn (i.e. both daytime and night-time during the 24-hour day), compared to a group receiving a placebo.

The method of the invention further provides a statistically significant increase in the proportion of nighttimes during a 14-day course of therapy that a randomized treatment group administered lansoprazole or its pharmaceutically acceptable salt is substantially free of heartburn, compared to a group receiving a placebo.

The patient is a mammal, for example, a human (e.g., an adult 18 years or older). The patient is preferably a human in need of treatment for upper gastrointestinal tract conditions.

### DETAILED DESCRIPTION OF THE INVENTION

The clinical symptom of heartburn is an upward moving, uncomfortable sensation behind the breastbone, frequently accompanied by a burning feeling. By “frequent heartburn” is meant heartburn occurring a minimum of 2 days a week. By “daytime heartburn” is meant heartburn that occurs between when the patient rises for the day and when the patient retires to sleep that night. By “nighttime” (or “nocturnal”) heartburn is meant heartburn occurring between when the patient retires to sleep (supine position) and when the patient rises the next day (upright position), which is a period of approximately 8 hours. “Frequent nighttime heartburn” may be defined as nighttime heartburn which has occurred on at least 2 days per week over the past month if the subject's heartburn was left untreated.

The present invention comprises a method of treating or preventing frequent heartburn in a patient in need thereof which comprises administering to the patient an effective amount of lansoprazole or a pharmaceutically acceptable salt thereof per day for a treatment duration of up to 14 days.

Preferably, the method comprises administering to the patient lansoprazole or a salt thereof in a sufficient amount to provide a dose of 15 mg lansoprazole (free acid) per day for up to 14 days.

Alternatively, the method comprises administering lansoprazole or a salt thereof in a sufficient amount to provide a dose of 30 mg lansoprazole (free acid) per day for up to 14 days.

In a preferred embodiment, the lansoprazole or pharmaceutically acceptable salt thereof is administered in a single daily dose for up to 14 consecutive days.

Most preferably, the lansoprazole or pharmaceutically acceptable salt thereof is administered once per day for 14 days. Said 14-day course of therapy is preferably carried out no more frequently than every 4 months. Thus the method preferably comprises administering lansoprazole 15 mg or 30 mg once per day for 14 days, and optionally repeating said 14-day course of therapy at intervals of at least 4 months during which no lansoprazole is administered.

Advantageously, the method of the invention provides statistically significant heartburn relief commencing within Days 1-4 of treatment, for example within Days 1-3 (i.e. on Day 3), and even more preferably within Days 1-2 (e.g., on Day 2), and most preferably on Day 1, of treating.

“Day 1” of treating shall mean the 24-hour period measured from the first administration of the daily dosage amount of lansoprazole.

Assuming a treatment regimen comprising once daily administration of the drug at breakfast, “Day 2” (and subsequent days) of treating shall mean the approximately 24-hour period measured from waking on the second day to the third dose of medication (et seq.).

In general, references herein to the condition of being “substantially free of heartburn” (or “substantially heartburn free”) shall mean that the patient experiences no heartburn or heartburn of only mild severity.

More particularly, if requested to select a number from 0 to 3 corresponding to the Intensity Scale Definitions set forth in Table 6, infra to describe the most severe episode of heartburn occurring during a given evaluation period, a patient rendered “substantially free of heartburn” would assign a value of 0 or 1, preferably 0.

The lansoprazole may be taken any time of day, but is preferably taken just prior to or with a meal. When administered as a single daily dose, it is preferably taken just prior to breakfast.

Lansoprazole is available in 15 and 30 mg oral capsules (delayed release pellets), orally disintegrating delayed release tablets, and oral delayed release suspension (as well as intravenous injection) from TAP Pharmaceuticals (Lake Forest, Ill.) under the tradename Prevacid®.

A multicenter, double-blind, randomized, parallel group, placebo-controlled clinical study is carried out to investigate the safety and efficacy of lansoprazole, 15 mg (or alternatively 30 mg) once daily, in preventing frequent heartburn.

A primary objective of the study is to demonstrate that repeated doses of 15 mg (or alternatively, 30 mg) of lansoprazole once a day are effective in increasing the proportion of 24-hour days substantially free of heartburn during the 14 days of treatment as compared to placebo.

A secondary objective is to demonstrate that repeated doses of 15 (or 30) mg of lansoprazole once a day provide statistically significant increases in the proportion of night-times with no heartburn during 14 (24-hour) days of treatment in patients so treated as compared to patients receiving a placebo.

A further objective is to demonstrate that the treatment regimen achieves full effect on Day 4 or Day 3 of treating, and more preferably on Day 2, and even on Day 1, of treating.

As illustrated in Table 1 below, the study design consists of 4 phases: (1) a one-week screening and heartburn medication wash out period, (2) a one-week placebo run-in period to allow evaluation of heartburn frequency and compliance of the subject with daily medication dosing and diary completion; (3) a placebo-controlled treatment period of two-weeks; and (4) a placebo follow-up period of one-week that allows assessment of the time to recurrence of heartburn.

TABLE 1

| ← Screening → | | ← Treatment → | | ← Follow-up → |
|---|---|---|---|---|
| Heartburn | ← Run-in → | Randomization | End of | End of single |
| medication | Single blind | (Double blind | double blind | blind placebo |
| wash out | placebo | treatment | treatment | follow-up |
| Study Day −14 | Study Day −8 | Study Day 1* | Study Day 16 | Study Day 24 |
| (±2) | (+4) | | (+4) | (+4) |
| Visit 1 | Visit 2 | Visit 3 | Visit 4 | Visit 5 |

*“Study Day 1” signifies the first day of randomization. All time-points in this study are relative to Study Day 1.

The trial population consists of subjects (male or female, at least 18 years old) with frequent heartburn, i.e. heartburn occurring a minimum of 2 or more days a week over the past month, that was untreated. A total of 576 subjects with frequent heartburn (288 per group) is randomized in order to yield 546 subjects.

The study population is divided equally between a treatment group receiving lansoprazole capsules, 15 mg (or alternatively, 30 mg) and a control group receiving placebo capsules. All statistical tests used to compare the two treatment groups are two-sided, with probability of Type I error=0.05.

Inclusion criteria are as indicated in Table 2:

TABLE 2
Inclusion Criteria:
At screening the subject must:

| | |
|---|---|
| 1 | Understand and provide written informed consent, understand the |
| | procedures, and demonstrate an ability to understand and follow |
| | diary instructions prior to initiation of any study procedure. |
| 2 | Have heartburn on at least two days per week over the past month |
| | if their heartburn was left untreated. |
| 3 | Have heartburn which responds to antacids or over-the-counter |
| | (OTC) H₂RA. |
| 4 | Have heartburn which responds completely to short term OTC PPI |
| | (e.g., Prilosec OTC ®) treatment consistent with the current OTC |
| | use (no more than a 14-day course every four months). |
| 5 | Be willing to discontinue the use of antacids, OTC H₂RA, and OTC |
| | treatment prior to the start of the run-in period. The washout is |
| | required to be a minimum of 1 day for antacids, or 7 days for an |
| | OTC H₂RA or OTC PPI. |
| 6 | Be a male or non-pregnant, non-lactating female, at least 18 |
| | years old. |

At run-in the subject must:

| | |
|---|---|
| 1 | Have completed a washout of a minimum of 1 day for antacids, or |
| | 7 days for an OTC H₂RA or OTC PPI (Prilosec OTC ®). |

At randomization the subject must:

| | |
|---|---|
| 1 | Have had the presence of heartburn on at least two days per week |
| | during the run-in phase. |
| 2 | Have had no more than one day with missed doses or with |
| | incomplete or inconsistent entries in the run-in diary. |
| 3 | Return for the baseline (randomization) visit (Visit 3) no more |
| | than two days after scheduled date. |

Exclusion criteria are as specified in Table 3:

TABLE 3
Exclusion Criteria
Subjects with:

History of erosive esophagitis verified by endoscopy.
History of gastroesophageal reflux disease diagnosed by a physician
and confirmed by endoscopy.
History of pathologic intra-esophageal pH monitoring.
PPI treatment that has been more than a 14-day course every four
months.
Any underlying medical condition or necessary concomitant medication
which may interfere with the evaluation of heartburn treatment.
Clinically significant and/or unstable renal or hepatic disease as
demonstrated via medical history or screening laboratory analyses.
The need for continuous treatment with histamine-2 receptor
antagonists or proton pump inhibitors or motility drugs. The previous
intermittent use of these antisecretory agents is permitted as long
as they are discontinued after the subject provides his/her consent
at the start of the run-in phase.
The need for continuous treatment with a prescription antifungal, or
warfarin.
An unwillingness to participate in this study by taking something
other than Gelusil ® antacid for the rescue treatment of acute
heartburn.
The use of any antacids for other indications (e.g., dyspepsia,
diarrhea, calcium supplement) throughout the study.
Known hypersensitivity to any component of lansoprazole or Gelusil ®
antacid.
Participation in another investigational drug study within 30 days of
the screening visit.
History (within the past 12 months) of alcoholism or illicit drug use
or abuse, or any condition associated with poor compliance.
Any other medical condition or situation that the investigator feels
constitutes a safety concern (e.g., gastrointestinal bleeding,
malignancy, etc.).
Females who are pregnant or lactating.

Lansoprazole delayed-release capsules, 15 mg (or alternatively, 30 mg), or placebo in identical appearing capsules, are packaged into aclar film/foil blister strips in a seven-dose configuration sealed in child resistant blister cards or in high density polyethylene containers with child resistant closures. The lansoprazole capsules are dispensed as follows (Table 4):

TABLE 4

| | |
|---|---|
| Run-in Visit (2) | Dispense blister card or bottle containing one |
| | aclar film/foil blister strip of study medication |
| | for a 1 week supply of study medication |
| Randomization Visit | Dispense blister card or bottle containing two |
| (3) | aclar film/foil blister strips of study medication |
| | for a 2 week supply of study medication |
| End of double blind | Dispense blister card or bottle containing one |
| treatment Visit (4) | aclar film/foil blister strip of study medication |
| | for a 1 week supply of study medication |

The study is double-blind during the treatment period and single-blind during the run-in.

The placebo capsule is taken orally once daily for 7 days during the single blind run-in period, and once daily for 7 days during the single blind follow-up period.

Lansoprazole 15 mg (or alternatively, 30 mg) capsules or placebo are taken once daily for 14 days during the double blind treatment period.

Patients are instructed that the capsules should be swallowed whole with a glass of water once a day before eating, preferably in the morning with breakfast, but in any case before noon.

Antacid Gelusil® tablets, Parke-Davis Pharmaceuticals (aluminum hydroxide 200 mg, magnesium hydroxide 200 mg, simethicone 15 mg),) are provided as rescue medication. Subjects are instructed that individual doses of 2-4 tablets may be taken according to the label instructions when necessary for the relief of acute heartburn, but no sooner than 1 hour after study medication has been taken; and no more than 12 tablets are to be taken a day. Subjects are encouraged not to use the rescue medication unless absolutely necessary.

Detailed instructions are provided to the subjects, as well as a diary for recording the time that study medication was taken. The evaluation schedule is as follows (Table 5):

TABLE 5
Evaluation schedule.

| | Phase | | | | |
|---|---|---|---|---|---|
| | Screening | Run-In | Randomization | Treatment | Follow-up |
| | Day(s) relative to Study Day 1 | | | | |
| | −14 (±2) | −8 (+4) | 1 | 16 (+4) | 24 (+4) |
| | Visit | | | | |
| Procedures | 1 | 2 | 3 | 4 | 5 |
| Written informed consent | X | | | | |
| Inclusion/exclusion criteria | X | X | X | | |
| Medical history | X | | | | |
| Physical exam | X | | | | |
| Vital signs | X | | | | |
| ECG | X | | | | |
| Laboratory Analysis | X | | | | |
| IVRS diary instructions | | X | X | X | |
| Prior/Concomitant medications | X | | X | X | X |
| Adverse events | | | X | X | X |
| Dispense study medication | | X | X | X | |
| Dispense rescue medication | X | X | X | X | |
| Study medication accountability | | | X | X | X |
| Telephone contact | | | | X | |
| Subject Global Assessment | | | | X | |
| Quality of Life | X | | | | |

### Screening (Visit 1)

At screening (Visit 1), a subject meeting all inclusion/exclusion criteria that can be evaluated at the screening visit is allocated a screening number. Demographic information and medical history are collected, including frequency and severity of heartburn episodes, smoking and alcohol consumption, and medication. The subject is asked by the investigator about how his/her heartburn affects his/her quality of life, and the response is recorded on the Visit 1 case report form (CRF). A physical exam is conducted including vital signs. An ECG is performed, blood is drawn for hematology and chemistry tests, and a urine pregnancy test is performed for women of childbearing potential. All pre-study heart-burn medications are stopped. The subject receives a supply of rescue medication.

### Placebo Run-In Period (Visit 2)

During the placebo run-in period, Interactive Voice Response System (IVRS) diaries are completed daily. At Visit 2, the subject is instructed in the proper completion of the diary. The subject receives a supply of study and rescue medication, and is instructed regarding appropriate use of medication and recordation of use in the IVRS diary. The subject is instructed not to take rescue medication within 1 hour of study medication and that it is to be used only if absolutely necessary for the treatment of acute heartburn.

The subject enters the run-in period (8+4 days), which must be a minimum of 7 days, in order to assess the frequency and severity of heartburn, IVRS diary completion compliance, and visit schedule compliance.

### Randomization (Visit 3)

At Visit 3, use of study and concomitant medications since the screening visit is recorded. Remaining study and rescue medication is collected. Information on adverse events (AEs) is collected (symptoms occurring before application of randomized study medications are captured as run-in AEs). Inclusion/Exclusion criteria are evaluated. The subject must meet the following randomization criteria: (1) heartburn at least two days per week during the Run-in phase; (2) no more than one day with missed doses or with incomplete or inconsistent entries in the IVRS Run-In diary; and (3) return for Visit 3 of no more than two days after the scheduled date. The subject is assigned the lowest available randomization number, and enters the treatment period. Study and rescue medication are dispensed to the subject. The subject is instructed again regarding appropriate use of medication and recordation of medication in the IVRS diary. The subject is reminded not to take rescue medication within one hour of study medication and that it is to be used only if absolutely necessary for the treatment of acute heartburn.

### End of Double Blind Treatment (Visit 4)

On day 16 (+4) following Visit 3 the subjects return for Visit 4 which must occur after a minimum of 14 days of treatment. Subjects are asked about their treatment satisfaction using the Global assessment questions (Table 8) in the Visit 4 CRF, and the responses are recorded on the CRF. The use of study and concomitant medications since the screening visit is recorded. The remaining study and rescue medication is collected. Information on AEs is recorded (symptoms occurring since Visit 3).

### End of Study (Visit 5)

On day 24 (+4) following Visit 4 the subjects return for Visit 5, which must occur after a minimum of 7 days of treatment. The use of study and concomitant medications since the screening visit is recorded. Remaining study and rescue medication is collected. Information on AEs is recorded (symptoms occurring since Visit 4).

### Efficacy Assessments

Most measures of efficacy are derived from data recorded in the subject's IVRS diary after a daily self-assessment. Subjects are given instructions to complete the IVRS diary in the morning for the previous 24-hour period on a daily basis for all phases of the study. The diary is designed to collect information on the occurrence of daytime and nighttime heartburn episodes, maximum heartburn severity, and rescue medication consumption for the previous 24 hours. In addition, the date and time of study medication consumption are recorded.

To classify heartburn severity, subjects are instructed to use the most intense episode of the 24-hour period according to the following Intensity Scale Definitions (Table 6):

TABLE 6
Intensity Scale Definitions.:

| | |
|---|---|
| 0 | No heartburn: No heartburn is present |
| 1 | Mild: Heartburn is present but easily tolerated |
| 2 | Moderate: Heartburn is sufficient to cause interference with normal |
| | daily activities or sleep |
| 3 | Severe: Heartburn is incapacitating. Subject is unable to perform |
| | normal daily activities or sleep |

Every morning during the study, each subject completes the diary by answering the following questions (Table 7):

TABLE 7
1. Daytime heartburn.
On Study Day 1: Did you experience heartburn anytime during the day
(from taking your study medication after getting out of bed to going
to bed)?
On Study Days 2-14: Did you experience heartburn anytime during the
day (from getting out of bed yesterday to going to bed)?
2. Nighttime heartburn.
Did you experience nighttime heartburn (from going to bed yesterday
to getting out of bed)?

Subjects are instructed to complete their IVRS diary and take their dose of study medication prior to breakfast, and ensure that the date and time are recorded in the IVRS diary.

In addition, subjects are asked by the investigator about their treatment satisfaction using the following subject global assessment questions (Table 8), and the responses are recorded on the Visit 4 CRF:

TABLE 8
Global assessment questions

| | |
|---|---|
| 1. | Overall, how satisfied have you been with the study medication |
| | you received for your heartburn during the last 2 weeks? |
| | (Possible responses: extremely satisfied, very satisfied, |
| | satisfied, satisfied just a bit, not at all satisfied.). |
| 2. | How much have you benefited from the study medication you |
| | received for your heartburn during the past 2 weeks? |
| | (Possible responses: not at all, a little bit, some, a lot.). |
| 3. | Would you recommend the study medication you received for your |
| | heartburn to someone else with your condition? (Possible |
| | responses: yes, no, unsure.). |
| 4. | Given your experience with other medications for your daytime |
| | heartburn, would you say your study medication provided? |
| | (Possible responses: much better relief, somewhat better relief, |
| | the same level of relief, somewhat less relief, much less relief). |
| 5. | Given your experience with other medications for your nighttime |
| | heartburn, would you say your study medication provided? |
| | (Possible responses: much better relief, somewhat better relief, |
| | the same level of relief, somewhat less relief, much less relief). |

### Statistical Methods

### Populations

The Intent-to-Treat (ITT) set includes all randomized subjects who are dispensed the study medication, and have at least one post-baseline efficacy assessment (diary recording of the occurrence and severity of heartburn). The analysis of the ITT is considered the primary efficacy analysis.

The per protocol set (PPS) includes subjects in the ITT who do not violate the protocol in any way liable to influence the efficacy outcome. The per protocol population is determined and documented after data base lock and before the study is unblinded. The only results presented for the PPS are the analyses of the primary efficacy endpoint.

### Background and Demographic Characteristics

Information collected at the Screening visit and in the subject diaries completed during the run-in phase is summarized for the purpose of characterizing the subject populations. This information includes background information (age, gender, race, and smoking habits), and medical history, including triggers for heartburn, quality of life measures, and concomitant medications. It also includes details of heartburn experienced during the run-in phase, including the occurrence of nighttime and daytime heartburn, severity, and the use of study medication and rescue medication, each recorded daily. As data is collected only for the most severe episode in a day, the severity will correspond to that most intense episode. Data collected at screening on physical exam, vital signs, ECG and safety laboratory variables are presented in subject listings.

Categorical variables (e.g., sex, presence/absence of relevant medical history, any previous/current medication, smoking habit, quality of life measures) are summarized by the number and percentage of subjects with each relevant characteristic in each treatment group. In addition, listings of medical conditions and previous/current medications are presented.

Continuous variables such as age are summarized by calculating the mean and standard deviation, and the median, minimum and maximum values in each treatment group.

The number of days on which heartburn (including nighttime, daytime, and 24 hour days) was experienced over the run-in phase are summarized by calculating the mean and standard deviation, and the median, minimum and maximum values in each treatment group both overall as well as for data normalized to 7 days.

The number and percentage of subjects in each treatment group who experience nighttime heartburn each day, and at least once during the run-in phase, are presented both overall as well as for data normalized to 7 days. Daytime heartburn is summarized similarly.

The severity of the most intense episode of daytime heartburn each day over the run-in phase, recorded on a 4-point ordinal scale (None, Mild, Moderate, Severe) is treated as a categorical variable for presentation purposes; the percentages of subjects with each severity grade are presented graphically. Similar analyses are presented for nighttime heartburn and for the most severe episode over each 24-hour day.

Use of study medication during the run-in phase is summarized with a frequency tabulation. Use of rescue medication is summarized by the number of days on which rescue medication was taken; this variable is treated as continuous and summarized appropriately.

All baseline data collected either at the screening visit or during the entire run-in phase are listed by treatment group, center and subject.

Homogeneity at baseline is examined between treatment groups. If a noteworthy difference is found, its effect on the efficacy comparisons is investigated and, as warranted by the data, terms are included in exploratory models to take any imbalances into account.

### Study Medication

Compliance with study medication, based on the study medication log, is analyzed separately for the run-in and double-blind treatment periods. For each period of the study, the number and percentage of days on which the subject took the study medication are summarized by calculating the mean, 95% confidence limits on the mean, and the median, minimum and maximum values for each treatment group. Use of study medication is also listed by treatment group, center and subject. For those subjects who complete the entire 14-day treatment period, descriptive statistics are presented for the number and percent of subjects who took between 80% and 100% of the scheduled dose regimen.

### Concomitant Medication

Use of other concomitant medication is summarized separately for the run-in and treatment periods. Medications are summarized by ATC Code, and the number and percentage of subjects in each treatment group who took a medication in each drug class, and any concomitant medication, during the relevant period (between Visit 0 and Visit 1, and between Visit 1 and Visit 2, respectively) are presented. Concomitant medications taken during the two periods are also listed by treatment group, center and subject.

### Primary Efficacy Variable

A primary objective of this study is to demonstrate that repeated daily doses of 15 mg of lansoprazole are effective in increasing the proportion of days with no heartburn during 14 days (24-hour days) of treatment as compared to placebo. Therefore, the primary evaluation is the period between the first dose (Day 1) through the end of the double blind treatment period, i.e. Days 1-14 for 24-hour days. “No heartburn over 24 hours” on each of Days 1-14 is defined as the combined experience of daytime and nighttime heartburn. On Day 1, evaluation of daytime heartburn begins with the first dose of study medication. On Days 2 through 14, evaluation of daytime heartburn begins upon rising from bed. The evaluation of nighttime heartburn ends with the next dose of study medication which is to be taken immediately after the subject has awoken and completed the diary.

The proportion of subjects in each treatment group with no heartburn on each day of the two-week double-blind treatment period is summarized and presented graphically. P-values for each day are presented based on an analysis using the Cochran-Mantel-Haenszel (CMH) test stratified by Center. In addition, the proportion of subjects in each treatment group with each total number of heartburn free days is presented.

The two treatment groups are compared by fitting an Analysis of Covariance (ANCOVA) to the percent of days with the indicated outcome using treatment and center as factors and the proportion of baseline heartburn days as a covariate. An analogous non-parametric ANCOVA is performed in support of the parametric procedure.

### Secondary Efficacy Variables

Secondary efficacy objectives are:
1. A comparison of treatment groups with regard to the proportion of nighttimes with no heartburn over the 14 day treatment period (proportion of days with no nighttime heartburn).

This variable is analyzed as the percentage of nighttimes with the outcome of no nighttime heartburn. The evaluation of nighttime heartburn begins upon going to bed and ends with the next dose of study medication which is to be taken immediately after the subject wakes and completes the diary.

The two treatment groups are compared by fitting an ANCOVA to the percent of days with the indicated outcome using treatment and center as factors and the proportion of baseline heartburn days as a covariate. An analogous non-parametric ANCOVA is performed in support of the parametric procedure.

2. A comparison of treatment groups with regard to the proportion of subjects with no heartburn during Day 1.

Efficacy on Day 1 is analyzed using the CMH test stratified by study center. The number and percentage of subjects who have no heartburn on Day 1 are presented by treatment group together with 95% confidence interval for each proportion. The difference in success rates between the two treatment groups will also be presented with its 95% confidence interval. As a secondary analysis of Day 1 efficacy, a logistic regression model is fitted and the Odds Ratio for the treatment effect are presented with its 95% confidence interval.

### Sample Size and Power Considerations

A total of 576 subjects are randomized in a 1:1 ratio (288 per group) to receive lansoprazole 15 mg or its matching placebo. The sample size is determined to detect a difference of approximately 15%, between lansoprazole 15 mg and placebo, in the percentage of days with the outcome of no heartburn over 24 hours over the first 14 days of dosing. A standard deviation of approximately 49.5% is taken to be adequate. A two group t-test for comparison of the treatment means with a 0.05 two-sided significance level has 90% power to detect an approximate 15% difference between placebo and lansoprazole 15 mg success rates when the sample size in each group is 230. The sample size allows for a conservative estimate of the combined early discontinuation and non-evaluability rate of 20%.

### RESULTS

Lansoprazole 15 mg once daily is superior in efficacy to placebo in the relief of frequent heartburn.

The invention is also directed to a kit comprising a container, written instructions for complying with a treatment regimen as described herein, and a plurality of dosage units of lansoprazole or a salt thereof.

Said written instructions are preferably directed to a method for treating frequent heartburn (i.e. occurring 2 or more days a week) and preventing heartburn from occurring during the course of treatment, including nighttime heartburn, comprising the steps of:

(a) providing to a patient in need thereof a course of therapy comprising the administration of a unit dose of lansoprazole (e.g., 15 mg or 30 mg, e.g. formulated with one or more pharmaceutically acceptable excipients into, e.g., a capsule or tablet) once per day for a period of 14 days; and

(b) thereafter observing a treatment-free period of at least 4 months during which no lansoprazole is administered; and

(c) optionally repeating the course of therapy of step (a) a second and optionally subsequent times, said second and any subsequent repetition being preceded by a treatment-free period as described in (b).

The kit may comprise a total of, e.g., 14, 28 or 42 unit dosage forms.

What is claimed is:

1. A method for treating or preventing frequent heartburn in a patient in need thereof comprising administering to the patient an effective amount of lansoprazole or a pharmaceutically acceptable salt thereof per day for a period of up to 14 days.

2. A method according to claim 1 comprising orally administering to the patient an effective amount of lansoprazole or a pharmaceutically acceptable salt thereof to provide 15 mg of lansoprazole per day for a period of up to 14 days.

3. A method according to claim 1 comprising orally administering to the patient an effective amount of lansoprazole or a pharmaceutically acceptable salt thereof to provide 30 mg of lansoprazole per day for a period of up to 14 days.

4. A method according to claim 2 comprising administering a daily dosage amount of 15 mg lansoprazole for 14 days.

5. A method according to claim 3 comprising administering a daily dosage amount of 30 mg lansoprazole for 14 days.

6. A method according to claim 4 wherein the patient so treated is substantially heartburn-free within Days 1-4 of treating.

7. A method of treating a patient susceptible to frequent heartburn to prevent or relieve nocturnal episodes thereof comprising administering to the patient lansoprazole or a pharmaceutically acceptable salt thereof in an effective amount per day for up to 14 consecutive days.

8. A method for treating frequent heartburn in a patient in need thereof, comprising the steps of
(a) providing to the patient a course of therapy comprising the administration of a unit dose of lansoprazole once per day for a period of 14 days; and
(b) thereafter observing a treatment-free period of at least 4 months during which no lansoprazole is administered; and
(c) optionally repeating the course of therapy of step (a) for a second and optionally subsequent times, said second and any subsequent repetition being preceded by a treatment-free period of at least 4 months.

9. A method according to claim 8 wherein the unit dose is 15 mg lansoprazole.

10. A method according to claim 8 wherein the unit dose is 30 mg lansoprazole.

11. A method according to claim 9 wherein the lansoprazole is in a capsule.

12. A method according to claim 10 wherein the lansoprazole is in a capsule.

13. A kit for treating frequent heartburn in a patient in need thereof comprising a container, written instructions for carrying out the method according to claim 8, and a plurality of unit doses of lansoprazole or a salt thereof.

14. A kit according to claim 13 comprising a total of, e.g., 14, 28 or 42 unit doses.
